(19) United States
(12) Patent Application Publication
(10) Pub. No.: US 2004/0028696 A1
(43) Pub. Date: Feb. 12, 2004

(54) Kefir as a potent anti-oxidant composition

(76) Inventors: Stan Kubow, Pointe-Claire (CA); Maryam Fotouhinia, Montreal (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/311,770

(22) PCT Filed: Jun. 18, 2001
(86) PCT No.: PCT/CA01/00899

(30) Foreign Application Priority Data
Jun. 22, 2000 (US) 60213268

(51) Int. Cl. A61K 35/78

(52) U.S. Cl. 424/195.16; 424/439; 424/750

(57) ABSTRACT

The present invention relates to an anti-oxidant composition having reducing effect on plasma lipid peroxidation and plasma TNF-α; concentrations in a subject, which comprises an effective anti-oxidant amount of an end-product of kefir manufacturing process for oral administration to the subject. The present invention also relates to a method of reducing plasma indices of lipid peroxidation and plasma tumor necrosis factor-α concentrations in a subject, which comprises administering orally an effective anti-oxidant amount of end-product of kefir manufacturing process to the subject. The present invention also relates to a prophylactic composition having neutraceutical properties, which comprises a neutraceutical effective amount of an end-product of kefir manufacturing process for oral administration to a subject.

### BACKGROUND OF THE INVENTION

(a) Field of the Invention

The invention relates to a novel anti-oxidant composition comprising kefir and therapeutical uses thereof.

(b) Description of Prior Art

Evidence has accumulated that hyperlipidemia is associated with a greater degree of oxidative stress as higher serum concentrations of lipid peroxides and lower concentrations of antioxidants have been described in patients with hyperlipidemia and atherosclerosis. Malondialdehyde, a marker of lipid peroxidation, was increased in the plasma of patients with congestive heart failure relative to controls (Diaz-Velez, C. R., 1996, Am Heart J 131:146-152). Increased oxidant stress can lead to the production of oxidized low-density lipoprotein, which is taken up by macrophages and deposited in fatty-streaks and early plaques. Although atherogenesis is a complex multistep progression, the formation and deposition of oxidized low-density lipoprotein appears to be a key early step in accelerated atherogenesis.

Immune mechanisms have been implicated to play an important atherogenic role as atherosclerosis is characterized by a chronic and excessive inflammatory response resulting from the trapping of low-density lipoprotein in the arterial wall. Recent studies have indicated that pro-inflammatory cytokines produced predominantly by activated macrophages and lymphocytes such as interleukin-1, tumor necrosis factor-α (TNF-α) and chemokines such as interleukin-8 rise in conditions of hyperlipidemia and atherosclerosis (Fernandez-Real, J. M et al., 1999, Diabetes 48:1108-1112). Although all of these cytokines play important roles as intercellular signals that recruit cells and mediate many inflammatory processes, TNF-α appears to be a critical mediator of the inflammatory cascade, and is also associated with the induction of hyperlipidemia and atherosclerosis (Fernandez-Real, J. M et al., 1999, Diabetes 48:1108-1112). Oxidative processes are involved in the induction of TNF-α as nuclear factor-KB (NF-KB), an oxidative stress sensitive transcription factor, controls the expression of a wide variety of genes active in inflammation that include cytokines such as TNFα (Barnes, P. J., Karin, M., 1997, N. Engl. J. Med. 336:1066-1071). Conversely, TNF-α has been shown to induce the production of reactive oxygen intermediates via respiratory burst in polymorphonuclear leukocytes (Tsujimoto, M., et al., 1986, Biochem. Biophys. Res. Commun. 137:1094-1100). There is increasing evidence that antioxidants have anti-inflammatory effects as antioxidants can block NF-KB activation. For instance, EGCG inhibits okadaic acid-induced TNFα production and gene expression in BALB/3T3 cells (Suganuma, M., et al., 1996, Cancer Res. 56: 3711-3715). Agents that have been suggested to reduce TNF-α include pentoxifylline, cyclosporine, many antioxidants, corticosteroids, anti-TNF monoclonal antibodies, recombinant TNF soluble receptors, and L-carnitine; however, most of these therapies have only been tested in vitro and so their efficacy to human clinical context is unclear.

There is increasing interest in the role of functional foods in health and disease. One such food is the fermented milk, kefir. Kefir can only be made from the incubation of milk with kefir grains and mother cultures prepared from kefir grains. Kefir grains contain a relatively stable and specific balance of microbes that include primarily lactic acid bacteria and yeast, which are held together by a matrix of polysaccharides. Kefir consumption is popular in Eastern Europe where it has had a long-standing tradition of health claims for the treatment of a variety of conditions including metabolic disorders, atherosclerosis, cancer and gastrointestinal disorders. Recently, lactobacillus GG and lactobacillus GG-fermented milk was shown to inhibit in vitro lipid peroxidation reactions and to act as powerful in vitro scavengers of superoxide anion (Ahotupa, M. et al., 1996, GG. Nutrition Today 31:51S-52S). Also, Zommara et al. (Zommara, M., et al., 1996, Nutr. Res. 16, 293-302; Zommara M., et al., 1998, Biosc. Biotech. Biochem. 62, 710-717) have demonstrated that the antioxidative effects of milk whey products are increased following bacterial fermentation. Also, yogurt formulations have recently been shown in the mouse model to reduce basal cytokine expression of several cytokine mRNAs, the depression of TNF-α mRNA being the most prominent effect.

In particular, it is not evident from previous work: (1) whether kefir exerts an anti-oxidative effect; and (2) whether kefir exerts any anti-inflammatory effect.

It would be highly desirable to be provided with an anti-oxidant composition comprising kefir and therapeutical uses thereof.

### SUMMARY OF THE INVENTION

One aim of the present invention is to provide an anti-oxidant composition comprising kefir and therapeutical uses thereof.

In accordance with the present invention there is provided an anti-oxidant composition having reducing effect on plasma lipid peroxidation and plasma TNF-α concentrations in a subject, which comprises an effective anti-oxidant amount of an end-product of kefir manufacturing process for oral administration to the subject.

The anti-oxidant composition in accordance with a preferred embodiment of the present invention, wherein the kefir end-product comprises a protein concentration of 48 mg protein/ml.

The anti-oxidant composition in accordance with a preferred embodiment of the present invention, wherein the subject is a human.

The anti-oxidant composition in accordance with a preferred embodiment of the present invention, wherein the kefir is manufactured using the kefir strain of Moscow.

The anti-oxidant composition in accordance with a preferred embodiment of the present invention, wherein the effective anti-oxidant amount is 500 ml/day.

In accordance with the present invention there is also provided a method of reducing plasma indices of lipid peroxidation and plasma tumor necrosis factor-α concentrations in a subject, which comprises administering orally an effective anti-oxidant amount of end-product of kefir manufacturing process to the subject.

The method in accordance with a preferred embodiment of the present invention, wherein the subject is a human.

The method in accordance with a preferred embodiment of the present invention, wherein the kefir is manufactured using the kefir strain of Moscow.

The method in accordance with a preferred embodiment of the present invention, wherein the effective anti-oxidant amount is 500 ml/day.

In accordance with the present invention there is also provided a prophylactic composition having neutraceutical properties, which comprises a neutraceutical effective amount of an end-product of kefir manufacturing process for oral administration to a subject.

The prophylactic composition in accordance with a preferred embodiment of the present invention, wherein the subject is a human.

The prophylactic composition in accordance with a preferred embodiment of the present invention, wherein the kefir is manufactured using the kefir strain of Moscow.

The prophylactic composition in accordance with a preferred embodiment of the present invention, wherein the effective anti-oxidant amount is 500 ml/day.

For the purpose of the present invention the following terms are defined below.

The term “kefir” is intended to mean an end-product of a kefir manufacturing process.

The term “subject” is intended to mean any mammals, including without limitation, human, canine, feline, equine, canine, bovine among others.

### BRIEF DESCRIPTION OF THE DRAWINGS

FIG. 1 illustrates the effects of kefir and milk intake on plasma TBARS concentrations over four weeks in a cross-over design study;

FIG. 2 illustrates the effects of kefir and milk intake on plasma TNF-alpha concentrations over four weeks in a cross-over design study; and

FIG. 3 illustrates a schematic representation of the kefir manufacture.

### DETAILED DESCRIPTION OF THE INVENTION

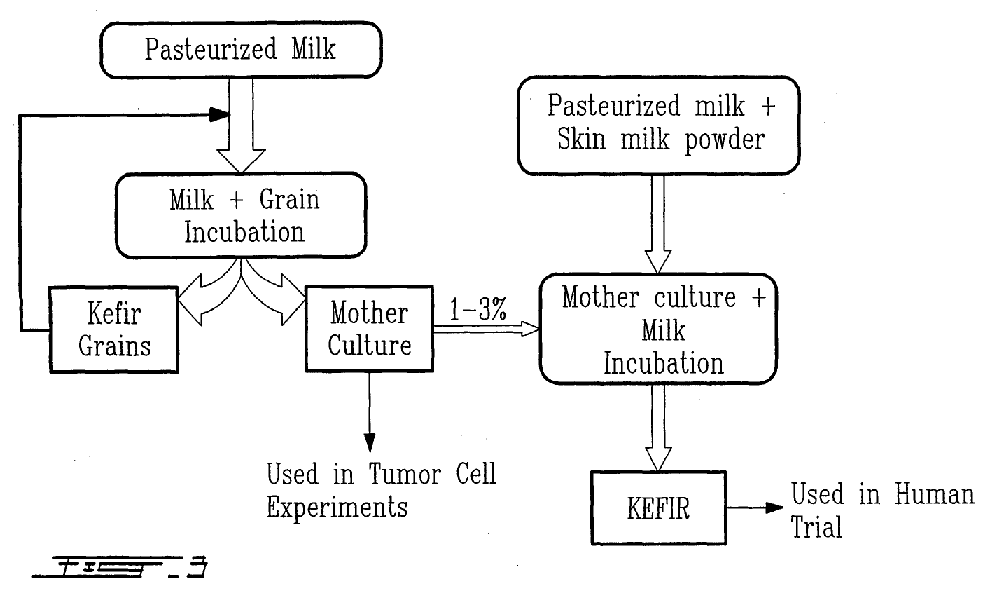

In accordance with the present invention, there is demonstrated for the first time that a kefir product is associated with a potent anti-oxidant effect. The kefir product is the end-product of the kefir manufacturing process as illustrated on FIG. 3. To determine the effects of kefir on plasma indices of lipid peroxidation and plasma tumor necrosis factor-α concentrations, 13 healthy mildly hypercholesterolemic men were given supplements of either 500 ml kefir or 500 ml milk for a period of four weeks as part of a randomized, placebo-controlled cross-over study with a four week intervening wash-out period. After 15 and 29 days of the kefir supplementation body weights were increased significantly (p<0.05) but body weight remained stable during the placebo phase. After 4 weeks of supplementation in both phases of the study, kefir intake was associated with significantly lower plasma concentrations of an index of lipid peroxidation, thiobarbituric acid reactive substances. Plasma concentrations of tumor necrosis factor-α were decreased significantly after two and four weeks of kefir supplementation in comparison to milk intake in the first and second phases of the feeding trial. Milk supplementation exerted no effect on oxidative stress parameters apart from a significant increase in tumor necrosis factor-α concentrations in the fourth week of milk intake of the first phase of the study. The present findings signify that kefir intake can exert potent antioxidant effects in a free-living hypercholesterolemic male population.

Materials and Methods

Subjects

Recruitment of male subjects with inclusion criteria of total serum cholesterol levels between 6 and 10 mmol/L was carried out through advertisements in local newspapers. Smokers and subjects with history of heart disease, previously diagnosed with diabetes, hypertension or hypothyroidism, or treated with cholesterol-lowering agents were excluded from the study. Exclusion factors also included lactose intolerance, alcohol intake of more than 10 drinks per week and reported use of antioxidant supplements unless their use has been discontinued for more than two months. Thirteen male subjects, ages 27 to 61 years (47±9 years), gave informed consent to participate in this study. Their body mass index (BMI) ranged from 26 to 38 kg/m² with a mean BMI of 30.2±4.4 kg/m².

Experimental Design

A randomized, crossover placebo-controlled trial was carried out in which the placebo consisted of milk product consisting of unfermented 2% milk in combination with skim milk powder and water. The test product, kefir, was obtained from Liberty Foods, Inc. (Candiac, Quebec, Canada). Both products were flavored daily with 60 g/serving of either peach or strawberry puree (Liberty Company, Candiac, Quebec). To ensure that both products had the same fat content and caloric value (Table 1), the placebo milk product was prepared daily by adding 90 mL of skim milk powder to 380 mL of 2% milk followed by the addition of 100 mL of water to have equal volume as kefir.

TABLE 1
Nutritive value of unflavored kefir and milk

| | Kefir | Milk |
|---|---|---|
| Energy (kcal) | 284 | 284 |
| Fat(g) | 7.6 | 7.6 |
| Protein (g) | 23.6 | 23.6 |
| Carbohydrate (g) | 31.2 | 32.0 |

The experimental periods consisted of a four-week duration separated by a four-week washout period. Subjects consuming self-selected diets were randomly assigned to the intake of supplements of either 500 ml kefir or 500 ml milk product for the first phase of the study. During the washout period subjects did not consume any kefir and discontinued the milk product supplementation. Subjects consumed the alternate dairy product in the second phase of the study. All subjects consumed the dairy supplements under supervision at the Mary Emily Clinical Nutrition Research Unit in Ste-Anne-de-Bellevue, Quebec. Breakfast foods were also available for the subjects along with the dairy supplements. Subjects picked up their rations of treatment or placebo product at the Mary Emily Clinical Nutrition Research Unit on Fridays to consume at home over the weekend. During the study, subjects were asked to maintain their habitual diet and exercise regimens to be consistent with that before the onset of the trial. A food frequency questionnaire was completed at the end of the washout period and after the second phase to assess for intake of yogurt for the preceding month. Blood samples of 20 ml each were taken on days 8, 15, 22, 28 before breakfast for determination of plasma concentrations of thiobarbituric acid reactive substances (TBARS) and TNF-α. All blood samples were centrifuged at 1500 rpm for 15 min. Plasma was stored at −80° C. until analysis. The Human Research Ethics Committee of McGill University approved the experimental procedures and protocol.

Data Analysis

TNF-α was measured by a enzyme-linked immunosorbent assay kit (Intergen, MD) has a sensitivity of 0.3 pg/ml TNF-α in serum. This allows the measurement of TNF-α in the normal range in human plasma/serum. The assay was performed according to the manufacturer's instructions and the microtiter plates were read at 450 nm with use of the Multiskan Plus (Flow Laboratories) microplate reader. Lipid peroxides were determined in the serum by measuring TBARS, expressed as nanomoles per ml plasma. Statistical analyses were performed using Statistical Analysis Software (SAS version 6.04, Cary, N.C.). Data were analyzed using two-level analysis of variance with repeated measures with time and treatment as variables. The correlation between plasma TBARS and TNF-α concentrations was analyzed using Pearson's correlation analysis.

Results

Both forms of supplementation were generally well tolerated with mild cramping and constipation or bloating being reported only within the first week of supplementation. Compared to baseline weight measurements, a significant increase in weight of 0.7 kg was reported on days 15 and 29 during kefir supplementation (p<0.05) whereas a decrease in body weight of 0.1 kg was seen during milk supplementation phase. No significant difference in yogurt intake during the months of washout and phase II was observed.

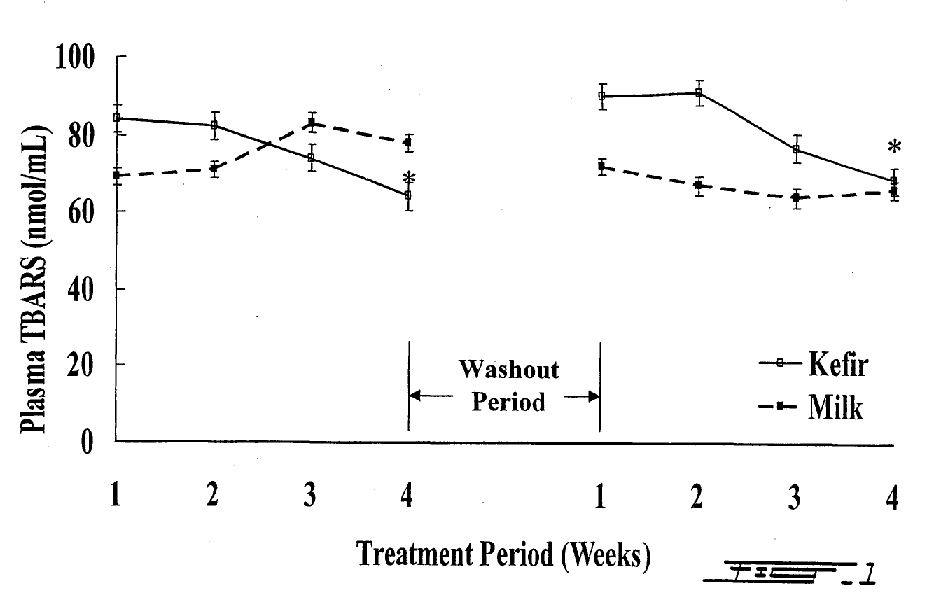

In phase I of the study the subjects receiving the kefir supplement showed a significant reduction in plasma concentrations of TBARS after 4 weeks of supplementation as compared to their plasma TBARS values after one and two weeks of kefir consumption (FIG. 1). Values are means±SEM and * denotes significantly different from weeks 2 and 3 at p<0.05. The subjects who started with milk supplementation did not show any significant changes in plasma TBARS concentrations; however, after the four week washout and a shift to kefir intake, a significant decrease in plasma TBARS concentrations was observed in the same subjects after four weeks of kefir intake in the second phase. Concurrently, the subjects who were originally fed kefir in the first phase of kefir feeding did not exhibit any plasma TBARS lowering effects after receiving the milk product supplement during the second phase of the study. Thus, subjects receiving the kefir supplement in both phases of the study demonstrated a significant reduction in TBARS concentrations.

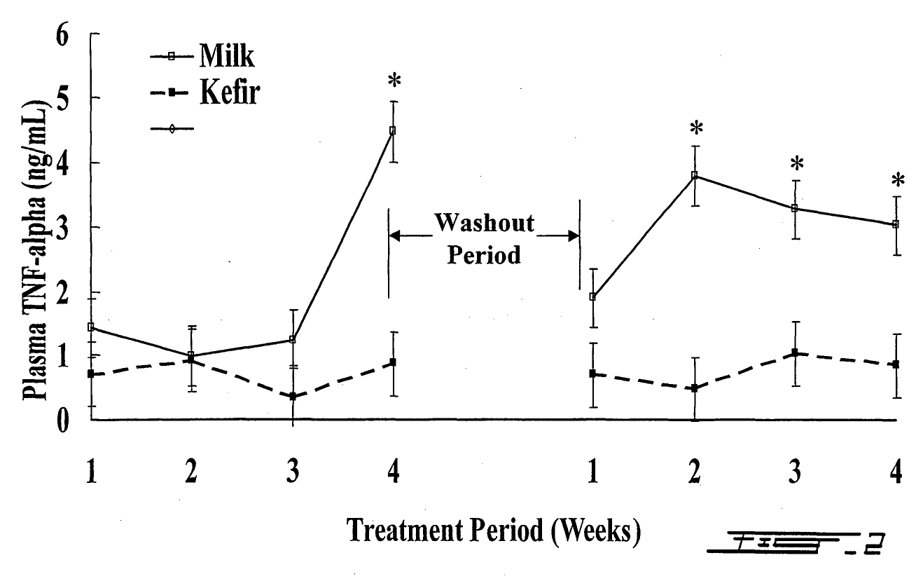

In terms of plasma concentrations of TNF-A the subjects showed a significantly lower plasma TNF-α concentrations after four weeks of kefir intake as compared to the milk-supplemented group (FIG. 2). Values are means±SEM and * denotes significantly different from kefir treatment at p<0.05. Milk intake was also associated with a significant increase in plasma TNF-α concentrations. After the 4-week washout period, kefir-fed subjects in the second trial phase showed significantly lower plasma TNF-α concentrations after two weeks of kefir intake whereas milk supplementation was not associated with a change in plasma TNF-α concentrations. A correlational analysis of plasma TBARS and TNF-α demonstrated a highly positive association (R=0.83; p<0.05).

Discussion

These results are the first to show that the consumption of a fermented milk product can exert antioxidative effects in humans. This study thus supports previous published in vitro and in vivo evidence that fermentation of milk products can exert antioxidative effects. Zommara et al. (Zommara, M., et al., 1996, Nutr. Res. 16, 293-302; Zommara M., et al., 1998, Biosc. Biotech. Biochem. 62, 710-717) showed that the antioxidative effects of the whey protein isolates was increased by bacterial fermentation. No previous work, however, has demonstrated that fermentation of milk via kefir grains can exert antioxidative properties. The mechanism by which kefir could induce an antioxidant action is unclear; however, milk peptides and lactic acid have been suggested as putative active antioxidative agents in fermented whey products (Zommara M., et al., 1998, Biosc. Biotech. Biochem. 62, 710-717).

This study also supports the fact that kefir consumption can block TNF-α production in humans as kefir intake showed lower plasma TNF-α concentrations relative to those observed with milk consumption. Due to an inadvertent loss of blood samples at baseline, however, the study cannot conclude on the relative effects of kefir intake to baseline TNF-α values. The highly significant positive correlation between plasma concentrations of TBARS and TNF-α, however, is supportive of the concept that the antioxidant effect associated with kefir intake exerted a reduction in TNF-α concentrations. This relationship is supported by previous studies indicating that antioxidants reduce inflammatory responses by attenuating NF-KB activation (Blackwell, T. S., et al., 1996, J Immunol. 157:1630-1637). It is unlikely that the antioxidant and anti-inflammatory action of kefir was the result of the small but significant weight gain (0.8%) observed during kefir consumption since overweight in human subjects is associated with enhanced risk of oxidative stress and elevated serum TNF-α concentrations.

Studies are contradictory regarding the effect of fermented milk products on TNF-α. The presence of yogurt bacteria resulted in the induction of human blood mononuclear cells cultured in produced interleukin 1β, TNF-α, and interferon α and γ (Solis-Pereyra, B., 1997, Am J Clin Nutr 66: 521S-525S). On the other hand, mice fed semi-purified diets containing either unheated yogurt or heat-treated yogurt showed a depression of basal gene expression of cytokines in systemic and mucosal sites.

In conclusion, in the present study, it was showed for the first time that daily kefir intake induces an antioxidant effect that correlates significantly with decreased plasma concentrations of TNF-α.

The present invention will be more readily understood by referring to the following examples which are given to illustrate the invention rather than to limit its scope.

### EXAMPLE I

### Prophylactic Composition

In accordance with one embodiment of the present invention, there is provided a prophylactic composition having neutraceutical properties, which comprises the end-product of the kefir manufacturing process. A preferred composition of this end-product of the kefir manufacturing process is 48 mg protein/ml.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An anti-oxidant composition having reducing effect on plasma lipid peroxidation and plasma TNF-α concentrations in a subject, which comprises an effective anti-oxidant amount of kefir for oral administration to said subject.

2. The anti-oxidant composition of claim 1, wherein said kefir comprises a protein concentration of 48 mg protein/ml.

3. The anti-oxidant composition of claim 1 or 2, wherein said subject is a human.

4. The anti-oxidant composition of claim 1, 2 or 3, wherein said kefir is manufactured using the kefir strain of Moscow.

5. The anti-oxidant composition of claim 3, wherein said effective anti-oxidant amount is 500 ml/day.

6. A method of reducing plasma indices of lipid peroxidation and plasma tumor necrosis factor-a concentrations in a subject, which comprises administering orally an effective anti-oxidant amount of kefir to said subject.

7. The method of. Claim 6, wherein said kefir comprises a protein concentration of 48 mg protein/ml.

8. The method of claim 6 or 7, wherein said subject is a human.

9. The method of claim 6, 7 or 8, wherein said kefir is manufactured using the kefir strain of Moscow.

10. The method of claim 6, 7, 8 or 9, wherein said effective anti-oxidant amount is 500 ml/day.

11. Use of an effective anti-oxidant amount of kefir for reducing plasma indices of lipid peroxidation and plasma tumor necrosis factor-α concentrations in a subject.

12. The use as claimed in claim 11, wherein said kefir comprises a protein concentration of 48 mg protein/ml.

13. The use as claimed in claim 11 or 12, wherein said subject in a human.

14. The use as claimed in claim 11, 12 or 13, wherein said kefir is manufactured using the kefir strain of Moscow.

15. The use as claimed in claim 11, 12, 13 or 14, wherein said effective anti-oxidant amount is 500 ml/day.

16. A prophylactic composition having neutraceutical properties, which comprises a neutraceutical effective amount of kefir for oral administration to a subject.

17. The prophylactic composition of claim 16, wherein said kefir comprises a protein concentration of 48 mg protein/ml.

18. The prophylactic composition of claim 16 or 17, wherein said subject is a human.

19. The prophylactic composition of claim 16, 17 or 18, wherein said kefir is manufactured using the kefir strain of Moscow.

20. The prophylactic composition of claim 16, 17, 18 or 19, wherein said effective anti-oxidant amount is 500 ml/day.
